(19) United States
(12) Patent Application Publication
(10) Pub. No.: US 2018/0228659 A1
(43) Pub. Date: Aug. 16, 2018

(54) LOW BASIS WEIGHT NONWOVEN WEB WITH VISIBLY DISTINCT PATTERNS

(71) Applicant: Kimberly-Clark Worldwide, Inc., Neenah, WI (US)

(72) Inventors: John H. Conrad, Alpharetta, GA (US); Mark G. Kupelian, Atlanta, GA (US); Eric E. Lennon, Roswell, GA (US); Kavon Pourasef, Marietta, GA (US)

(21) Appl. No.: 15/749,425

(22) PCT Filed: Aug. 31, 2015
(86) PCT No.: PCT/US2015/047815

§ 371 Date: Jan. 31, 2018

(51) Int. Cl. A61F 13/15; B32B 5/02; B32B 5/26; D04H 1/559; D04H 1/56; B32B 5/14; D04H 1/495; D04H 1/498; D04H 3/16; A61F 13/511; A61F 13/514

(52) U.S. Cl. CPC A61F 13/15203; A61F 2013/51338; B32B 5/26; D04H 1/559; D04H 1/56; B32B 5/142; D04H 1/495; D04H 1/498; D04H 3/16; B32B 5/028; A61F 13/15699; A61F 13/51121; A61F 13/51478; A61F 13/51401; B32B 2555/00; B32B 2307/726; D10B 2509/026; A61F 2013/15406; B32B 5/022

(57) ABSTRACT

The present invention relates to a SMS nonwoven laminate having a basis weight of less than about 20 gsm, said nonwoven laminate comprising outer layers of continuous spunbond filaments and an internal layer of from about 18% to about 43%, by weight of the nonwoven laminate of meltblown fibers, said nonwoven laminate having been hydroentangled to create visibly distinct patterns on the surface of at least one of the outer layers of said nonwoven laminate; and wherein prior to said hydroentangling, the content of meltblown fibers in the nonwoven laminate was from about 20% to about 45%, by weight of the nonwoven laminate.

### FIELD OF THE INVENTION

The present invention relates to a low basis weight nonwoven web possessing a visibly, distinct pattern and uncompromised integrity.

### BACKGROUND OF THE INVENTION

Delivering patterns or graphics on nonwoven webs helps to differentiate various materials and/or products. To deliver high quality graphics, however, costly printing may be used. In order to showcase patterns elevated basis weights are often used within the materials to deliver the contrast needed to be plainly visible to the consumer. Because of the high costs in ink or converting equipment, both of these options are not sustainable to manufacture an economically viable and attractive product. Additionally, the structural integrity of the product is important for overall quality and design.

Thus, it is desirable to deliver a nonwoven fabric material with a clearly perceptible pattern without an increase in basis weight and without compromising quality or integrity to the fabric.

### SUMMARY OF THE INVENTION

The present invention relates to a SMS nonwoven laminate having a basis weight of less than about 20 gsm, said nonwoven laminate comprising outer layers of continuous spunbond filaments and an internal layer of from about 18% to about 43%, by weight of the nonwoven laminate of meltblown fibers, said nonwoven laminate having been hydroentangled to create visibly distinct patterns on the surface of at least one of the outer layers of said nonwoven laminate; and wherein prior to said hydroentangling, the content of meltblown fibers in the nonwoven laminate was from about 20% to about 45%, by weight of the nonwoven laminate.

The present invention also provides for a personal care product wherein the product is an absorbent article comprising a SMS nonwoven laminate having a basis weight of less than about 20 gsm, said nonwoven laminate comprising outer layers of continuous spunbond filaments and an internal layer of from about 18% to about 43%, by weight of the nonwoven laminate of meltblown fibers, said nonwoven laminate having been hydroentangled to create visibly distinct patterns on the surface of at least one of the outer layers of said nonwoven laminate; and wherein prior to said hydroentangling, the content of meltblown fibers in the nonwoven laminate was from about 20% to about 45%, by weight of the nonwoven laminate.

The present invention also relates to a method of producing a SMS nonwoven laminate having a visibly distinct pattern and a basis weight of less than about 20 gsm, said method comprising the steps of: a) providing, in the machine direction to a hydroentangling equipment, a nonwoven laminate having a basis weight of from about 5 gsm to about 20 gsm, said nonwoven laminate comprising outer layers of continuous spunbond filaments and an internal layer of from about 20% to about 45%, by weight of the nonwoven laminate of meltblown fibers, to a web carrier surface of said hydroentangling equipment; b) continuing said nonwoven laminate on to a patterned screen roll wherein at least one manifold, of the hydroentangling equipment, comprises at least one water pressure jet positioned from about 0.005 m to about 0.05 m above said laminate and patterned screen roll; c) hydroentangling said nonwoven laminate with said jet at a water pressure of from about 200 psi to about 6000 psi; d) continuing said nonwoven laminate to a dryer; and e) providing a finished nonwoven laminate material having a basis weight of from about 5 gsm to about 20 gsm, wherein said nonwoven laminate material comprises outer layers of continuous spunbond filaments and an internal layer of from about 18% to about 43%, by weight of the nonwoven laminate of meltblown fibers, and wherein the surface of said nonwoven laminate material has a visibly distinct pattern as a result of the hydroentangling on the surface of at least one of the outer layers of said nonwoven laminate.

The present invention also relates to a packaging option wherein at least two distinct absorbent articles are contained within a package wherein both articles comprise a SMS nonwoven laminate having a basis weight of less than about 20 gsm, said nonwoven laminate comprising outer layers of continuous spunbond filaments and an internal layer of from about 18% to about 43%, by weight of the nonwoven laminate of meltblown fibers, said nonwoven laminate having been hydroentangled to create visibly distinct patterns on the surface of at least one of the outer layers of said nonwoven laminate; and wherein prior to said hydroentangling, the content of meltblown fibers in the nonwoven laminate was from about 20% to about 45%, by weight of the nonwoven laminate; however, the two articles are distinguishable from each other because the surface of one absorbent article possesses a visibly distinct pattern on at least one surface that is different than the visibly distinct pattern on at least one surface of the other absorbent article.

### BRIEF DESCRIPTION OF THE DRAWINGS

A full and enabling disclosure of the present invention is set forth more particularly in the remainder of the specification, including reference to the accompanying figures, in which:

FIG. 1 is a schematic diagram of one type of apparatus used in the manufacturing process, generally along the machine direction, in accordance with the present invention.

FIG. 2 is an enlarged view of a screen wire roll and manifold used in the manufacturing process in accordance with the present invention.

FIG. 3 is a photo image of two SMS nonwoven laminates side-by-side wherein the left SMS nonwoven laminate has not been hydroentangled according to the present invention and the right exemplifies a SMS nonwoven laminate according to the present invention with a visibly distinct pattern of small circles.

FIG. 4 is a photo image of two SMS nonwoven laminates side-by-side wherein the left SMS nonwoven laminate has not been hydroentangled according to the present invention and the right exemplifies a SMS nonwoven laminate according to the present invention with a visibly distinct wavy pattern. FIG. 5 is a photo image of one portion of a patterned screen used to produce the nonwoven laminate fabric of the present invention.

FIG. 6 is a photo image of the nonwoven laminate fabric of the present invention utilizing a patterned screen roll that incorporates the patterned screen shown in FIG. 5.

FIG. 7 is a photo image of one portion of a patterned screen used to produce the nonwoven laminate fabric of the present invention.

FIG. 8 is a photo image of the nonwoven laminate fabric of the present invention utilizing a patterned screen roll that incorporates the patterned screen shown in FIG. 7.

FIG. 9 is a SEM photo of a 17 gsm basis weight SMS nonwoven laminate comprising 70% spunbond and 30% meltblown that has not been produced by the process of the present invention. FIG. 10 is a SEM photo of a 17 gsm basis weight SMS nonwoven laminate comprising 70% spunbond and 30% meltblown that has been produced by the process of the present invention using 2 hydro jets at about 1450 psi each.

FIG. 11 is a SEM photo of a 17 gsm basis weight SMS nonwoven laminate comprising 70% spunbond and 30% meltblown that has been produced by the process of the present invention using 2 hydro jets at about 3481 psi.

FIG. 12 is a SEM photo of a cross-sectional view of a 17 gsm basis weight SMS nonwoven laminate comprising 80% spunbond and 20% meltblown that has been produced by the process of the present invention using 2 hydro jets at about 2901 psi.

FIG. 13 is a photo image of one portion of a patterned screen used to produce the nonwoven laminate fabric of the present invention.

FIG. 14 is a photo image of one portion of a three-dimensional patterned screen used to produce the nonwoven laminate fabric of the present invention.

### DETAILED DESCRIPTION OF THE INVENTION

While the specification concludes with the claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore; do not include solvents or by-products that may be included in commercially available materials, unless otherwise specified. The term “weight percent” may be denoted as “wt. %” herein. Except where specific examples of actual measured values are presented, numerical values referred to herein should be considered to be qualified by the word “about”.

As used herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The use of any trademarks herein has been noted with CAPITALIZATION of the word wherever it appears to acknowledge and respect the proprietary nature held by the owners of the mark. The word is followed by the generic terminology only wherever it appears for the first time herein.

As used herein the term “nonwoven fabric or web” refers to a web having a structure of individual fibers or threads which are interlaid, but not in an identifiable manner as in a knitted fabric. Nonwoven fabrics or webs have been formed from many processes such as for example, meltblowing processes, spunbonding processes, bonded carded web processes, etc.

As used herein, the term “meltblown web” generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin, et al., which is incorporated herein in its entirety by reference thereto. Generally speaking, meltblown fibers may be microfibers that are substantially continuous or discontinuous, generally smaller than 10 microns in diameter, and generally tacky when deposited onto a collecting surface.

As used herein, the term “spunbond web” generally refers to a web containing substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., U.S. Pat. No. 4,340,563 to Appel, et al. and U.S. Pat. No. 5,382,400 to Pike, et al., which are incorporated herein in their entirety by reference hereto thereto. Spunbond fibers are generally not tacky when they are deposited onto a collecting surface. Spunbond fibers may sometimes have diameters less than about 40 microns.

As used herein the terms “machine direction” or “MD” generally refers to the direction in which a material is produced. It is also often the direction of travel of the supportive moving carrier surface onto which fibers are deposited during formation of a non-woven web. The term “cross-machine direction” or “CD” refers to the direction perpendicular to the machine direction. Dimensions measured in the cross-machine direction (CD) are referred to as “width” dimensions, while dimensions measured in the machine direction (MD) are referred to as “length” dimensions. The width and length dimensions of a planar sheet make up the X and Y directions of the sheet. The dimension in the depth direction of a planar sheet is also referred to as the Z-direction.

As used herein, the terms “fluid entangling” and “fluid-entangled” generally refer to a formation process for creating a degree of fiber entanglement within a given fibrous nonwoven web or between fibrous nonwoven webs and other materials so as to move the fibers away from the web or from one area of the web to another. This may also ultimately impact the overall formation and make the separation of the individual fibers and/or the layers more difficult as a result of the entanglement. Generally, this is accomplished by supporting the web or other material on some type of forming or supportive moving carrier surface, such as a forming wire, which has at least some degree of permeability to the impinging pressurized fluid. A pressurized fluid stream (usually multiple streams at a manifold or series of manifolds) is then directed against the surface of the web and/or other materials which is opposite the supported surface of the web and/or other materials. The supported surface of a web and/or other material is also known as the wire or belt side, and the unsupported surface of a web and/or other material is also known as the material side. The pressurized fluid contacts the fibers of the web and forces portions of the fibers in the direction of the fluid flow, thus displacing all or a portion of a plurality of the fibers towards the supported surface (wire side) of the web. The result is a further entanglement of the fibers in what can be termed the Z-direction of the web (its depth direction or thickness). The degree of entanglement will depend on a number of factors including, but not limited to the degree of entanglement of the web or webs prior to subjection to the fluid entangling process, the type of fluid being used (liquids, such as water, steam or gases, such as air), the pressure of the fluid, the number of fluid streams, the speed of the process, the dwell time of the fluid and the porosity of the web or webs/other layers and the forming/carrier surface (such as a forming wire). One of the most common fluid entangling processes is that which is used to form the present invention, commonly referred to as hydroentangling. This is a well-known process to those of ordinary skill in the art of nonwoven webs with examples found in U.S. Pat. No. 3,485,706 to Evans, and U.S. Pat. Nos. 4,939,016, 4,959,531 and 4,970,104 to Radwanski. A typical hydroentangling process utilizes high pressure jet streams of water to entangle fibers to form a highly entangled consolidated fibrous structure. The hydroentangling manufacturing conditions described in such references are representative of operating conditions that are acceptable for use in manufacturing hydroentangled webs in accordance with the invention, unless otherwise noted. For the purposes of this application the abbreviation “HET'd” may be used as a shorthand notation for hydroentangled.

As used herein, the term “hydrophilic” generally refers to fibers or films, or the surfaces of fibers or films which are wettable by aqueous liquids in contact with the fibers. The term “hydrophobic” includes those materials that are not hydrophilic as defined. The phrase “naturally hydrophobic” refers to those materials that are hydrophobic in their chemical composition state without additives or treatments affecting the hydrophobicity.

As used herein, the term “g/cc” generally refers to grams per cubic centimeter.

The degree of wetting of the materials can, in turn, be described in terms of the contact angles and the surface tensions of the liquids and materials involved. Equipment and techniques suitable for measuring the wettability of particular fiber materials or blends of fiber materials can be provided by the Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “SMS” is the abbreviation for spunbond-meltblown-spunbond and is used to refer to the material that is a subject of the present invention.

As used herein, “SMS fabric laminate” is the low basis weight, precursor or starting SMS material that is prebonded or at least partially entangled prior to being further processed through the high pressure hydroentangling process of the present invention.

As used herein, “SMS patterned laminate” refers to the low basis weight, SMS fabric laminate comprising visibly distinct patterns as a result of high pressure hydroentangling.

The term “SMS web” is used to describe generally either the SMS fabric laminate or the SMS patterned laminate.

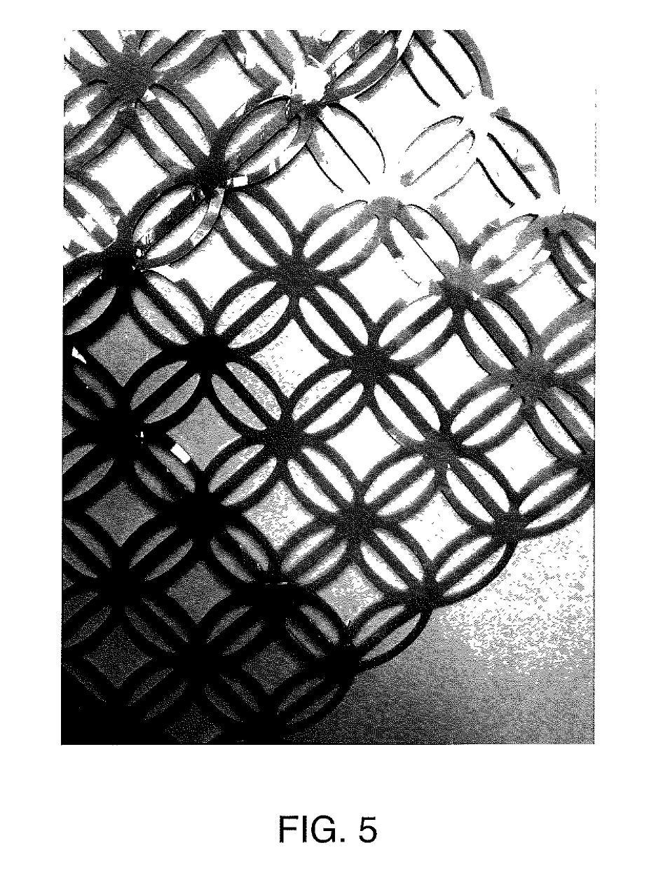

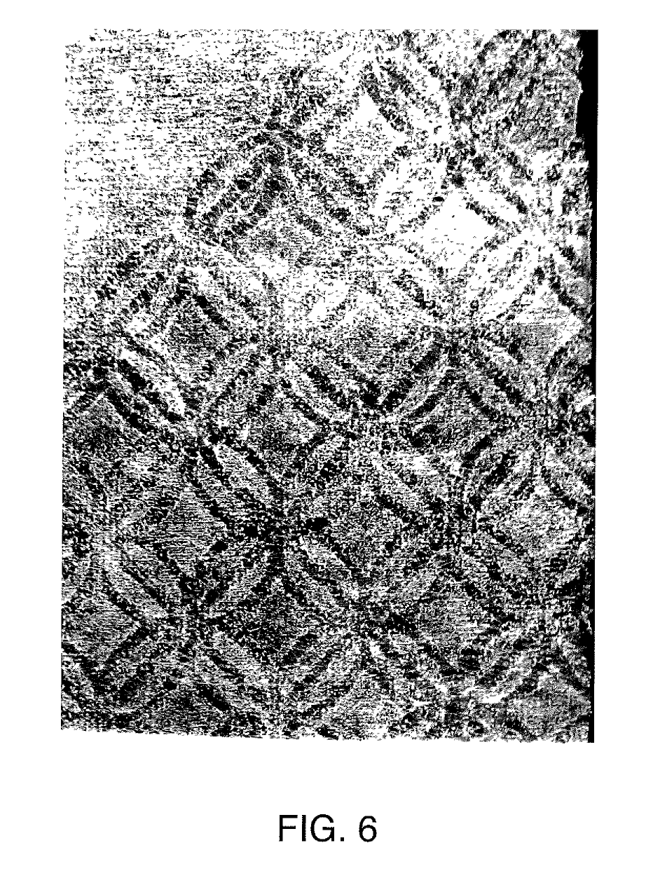

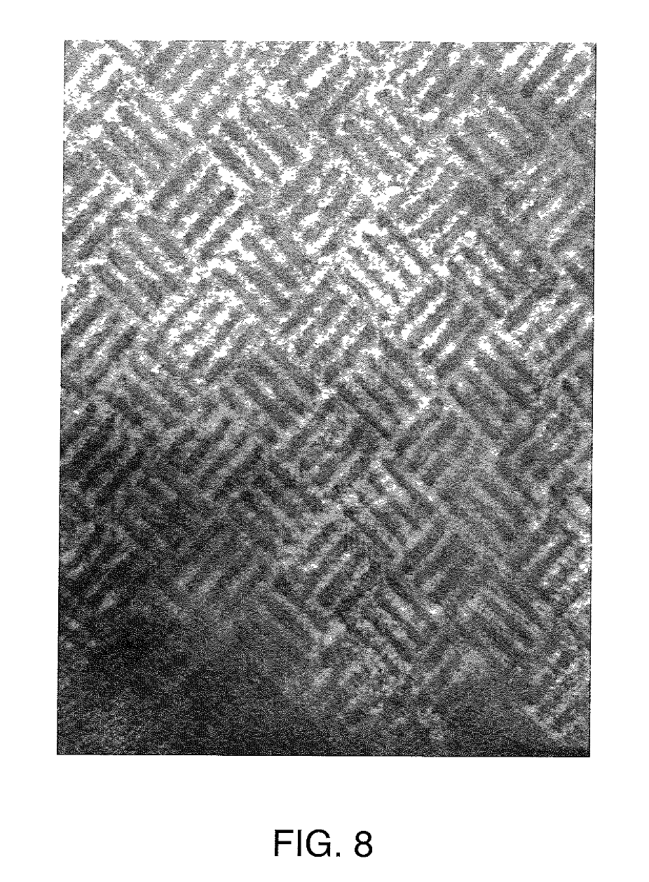

The term “patterned screen” refers generally to a flat wire mesh, three-dimensional wire mesh or a stencil-like metal surface with opening and closings such as those seen in FIGS. 6 and 8. A patterned screen is used on a flatbed belt or a drum within the hydroentangling equipment to create the visibly distinct SMS patterned laminate of the present invention. The visibly distinct pattern can be three-dimensional to give a textured-like surface area.

As used herein, the term “personal care product” includes absorbent articles such as diapers, training pants, absorbent underpants, adult incontinence products, sanitary wipes and feminine hygiene products, such as sanitary napkins, pads, and liners, and the like. The term “absorbent medical product” is used to refer to products such as medical bandages, tampons intended for medical, dental, surgical, and/or nasal use, surgical drapes and garments, coverings in medical settings, and the like.

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus it is intended that the present invention cover such modifications and variations.

### Spunbond

Spunbond nonwoven webs are made from melt-spun filaments. As used herein, the term “melt-spun filaments” refers to small diameter fibers and/or filaments which are formed by extruding a molten thermoplastic material as filaments from a plurality of fine, usually circular, capillaries of a spinnerette with the diameter of the extruded filaments then being rapidly reduced, for example, by non-eductive or eductive fluid-drawing or other well-known spunbonding mechanisms. The production of spunbond nonwoven webs is described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat No. 3,502,763 to Hartmann, U.S. Pat. No. 3,276,944 to Levy, U.S. Pat. No. 3,502,538 to Peterson, and U.S. Pat. No. 3,542,615 to Dobo et al. The melt-spun filaments formed by the spunbond process are generally continuous and have diameters larger than 7 microns, more particularly, between about 10 and about 30 microns. The spunbond filaments usually are deposited onto a moving foraminous belt or forming wire where they form a web. Spunbond filaments generally are not tacky when they are deposited onto the collecting surface.

Spunbond materials typically are stabilized or consolidated (pre-bonded) in some manner immediately as they are produced in order to give the web sufficient integrity to withstand the rigors of further processing into a finished product. This stabilization (pre-bonding) step may be accomplished through the use of an adhesive applied to the filaments as a liquid or powder which may be heat activated, or more commonly, by compaction rolls. As used herein, the term “compaction rolls” means a set of rollers above and below the web used to compact the web as a way of treating a just produced, melt-spun filament, particularly spunbond web, in order to give the web sufficient integrity for further processing, but not the relatively strong bonding of secondary bonding processes, such as through-air bonding, thermal bonding, ultrasonic bonding and the like. Compaction rolls slightly squeeze the web in order to increase its self-adherence and thereby its integrity. An alternative means for performing the pre-bonding step employs a hot air knife, as described in detail in U.S. Pat. No. 5,707,468 to Arnold et al.

The spunbond filaments of the present invention may comprise low loft, high loft or a combination thereof such as, but not limited to, that which is described in US Patent Application 2004/0077247 to Schmidt et al.

### Meltblown

Meltblown nonwoven webs are known in the art and have been used in a wide variety of applications. In general, meltblown fibers contained in meltblown webs have an average fiber diameter of up to about 10 microns with very few, if any, of the fibers exceeding 10 microns in diameter. Meltblown fibers are formed by extruding molten thermoplastic material through a plurality of fine, usually circular, die capillaries as molten threads or filaments into opposing flows of high velocity, usually heated gas streams, such as air, which attenuate the filaments of molten thermoplastic material to reduce their diameters and break the streams into discontinuous fibers of small diameter. Thereafter, the meltblown fibers are deposited onto a collecting surface to form a web of randomly dispersed meltblown fibers. The meltblown web possesses integrity due to entanglement of individual fibers in the web as well as some degree of thermal or self-bonding between the fibers, particularly when collection is effected only a short distance after extrusion. An exemplary process is disclosed in U.S. Pat. No. 3,849,241 to Butin et al., where air-borne fibers, which are not fully quenched, are carried by a high velocity gas stream and deposited on a collecting surface to form a web of randomly dispersed and autogenously bonded meltblown fibers. As is known in the art, the flow rate, temperature and pressure of the high velocity gas stream can be adjusted to form continuous meltblown fibers or discontinuous fibers. In addition, the flow rate, temperature and pressure of the high velocity gas stream can be adjusted to change the average fiber diameter and other properties of the fibers. Another process for forming a melt-blown web may be in accordance with U.S. Pat. No. 5,213,881 to Timmons et al., dated May 25, 1993. The meltblown nonwoven web may be formed using a single meltblown die or a series of meltblown dies. As is known in the art, the characteristics of the meltblown fibers can be adjusted by manipulation of the various process parameters used for each extruder and die head in carrying out the meltblowing process. The present invention provides a starting SMS fabric laminate that is to be fed into a hydroentangling equipment wherein the meltblown content is from about 20%, from about 25%, from about 28%, or from about 30% to about 35%, to about 40%, or to about 45%, by weight of the SMS fabric laminate prior to hydroentangling. After being processed through the hydroentangling equipment, the resultant SMS patterned composite comprises at least about 2% less meltblown fibers than the starting SMS fabric laminate material.

### SMS

SMS fabric laminates have outside spunbonded layers which are durable and an internal melt-blown barrier layer which may be porous. The meltblown fibers are randomly deposited on top of the spunbond layer to form a meltblown internal layer. The resulting SMS fabric laminate is then fed through bonding rolls. The bonding rolls are heated to the softening temperature of the polymer used to form the layers of the web. As the web passes between the heated bonding rolls, the material is compressed and heated by the bonding rolls, bonding each layer to layer with respect to the particular filaments and/or fibers within each layer. Bonding of SMS fabric laminates is well-known in the art and can be carried out as described by, for example, heated rolls or by means of ultrasonic heating of the web to produced thermally bonded filaments, fibers, and layers. In accordance with a conventional practice described in Brock et al., U.S. Pat. No. 4,041,203, the fibers of the meltblown layer in the fabric laminate can fuse within the bond areas while the filaments of the spun-bonded layers retain their integrity in order to achieve good strength characteristics. In accordance with the invention, the total basis weight of the SMS fabric laminate prior to the hydroentangling process is in the range generally of from about 5 gsm or from about 10 gsm to about 15 gsm or to about 20 gsm. The present invention presents a lightweight nonwoven laminate that is not typical as a starting material for incorporating patterns or designs within a fabric. Typically, embossing or pattern rolling, for example, may be used to create visibly distinct patterns in a nonwoven material. Thus, heavier materials such as pulp or nonwoven materials with heavy basis weights are typically used. Basis weights of previous SMS fabric laminates that exhibit visibly distinct patterns typically fall within a range of 25 gsm and above 34 gsm and above, 50 gsm and above and even as high as 500 gsm and above. Thus, the advantages of the present invention are well outside the scope of pre-existing patterned nonwoven materials and may replace such high basis weight woven textiles at a significantly lower cost.

### Hydroentangling Process

Hydroentangled nonwoven webs are known for exhibiting properties such as softness, high drape and comfort due to the increased fiber entanglement that leads to increased strength without an increase in shear modulus. The softness of the fabric can be explained by the fact that the entangled structures are more compressed than bonded ones. Additionally, there is more mobility and partial alignment of the fibers in the CD direction.

The present invention provides a method of producing a SMS nonwoven laminate material having a visibly distinct hydroentangled pattern and a basis weight of less than about 20 gsm by the steps of: a) providing, in the machine direction to a hydroentangling equipment, a nonwoven laminate having a basis weight of from about 5 gsm to about 20 gsm, said nonwoven laminate comprising outer layers of continuous spunbond filaments and an internal layer of from about 20% to about 45%, by weight of the nonwoven laminate of meltblown fibers, to a web carrier surface of the hydroentangling equipment; b) continuing said nonwoven laminate on to a patterned screen roll wherein at least one manifold, of the hydroentangling equipment, comprises at least one water pressure jet positioned from about 0.005 m to about 0.05 m above said laminate and patterned screen roll; c) hydroentangling said nonwoven laminate with said jet at a water pressure of from about 200 psi to about 6000 psi; d) continuing said nonwoven laminate to a dryer; and e) providing a finished nonwoven laminate material having a basis weight of from about 5 gsm to about 20 gsm, wherein said nonwoven laminate comprises outer layers of continuous spunbond filaments and an internal layer of from about 18% to about 43%, by weight of the nonwoven laminate of meltblown fibers, and wherein the surface of said nonwoven laminate material has a visibly distinct pattern on the surface of at least one of the outer layers of said nonwoven laminate.

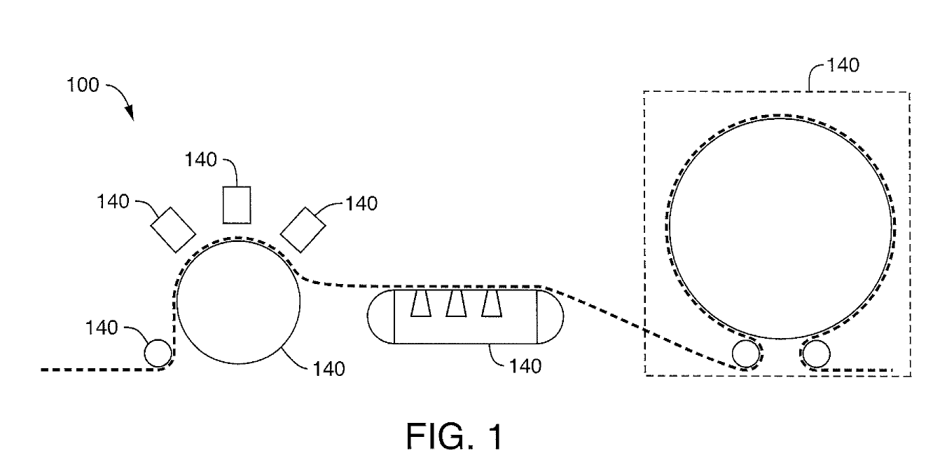

Referring to FIG. 1, the SMS fabric laminate (not shown) of the present invention may be prebonded or at least partially entangled and may be fed onto a supportive moving carrier surface (110) of the hydroentangling apparatus (100) directly from an SMS bonding equipment or it may be fed from a roll of wound SMS fabric laminate material. The SMS fabric laminate is then further treated through a high pressure hydroentangling process whereby it is moved onto a patterned screen roll (120) having a surface that is either flat, three-dimensional or a combination thereof. During hydroentangling, the meltblown fibers contained within the SMS fabric laminate are rearranged or in some areas forced away entirely in order to impart a pattern to at least one side of the SMS patterned laminate (not shown) by one or more jets within a manifold (130) or one or more jets within a succession of manifolds (130) (as shown with three manifolds). In some instances, both sides of the SMS fabric laminate may be subjected to hydroentangling.

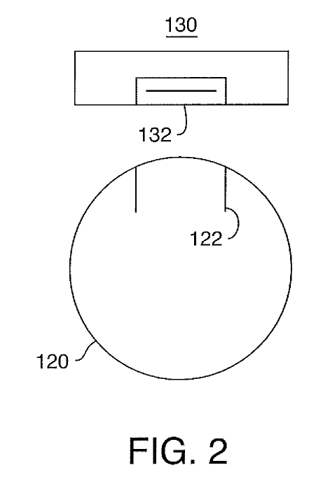

The hydroentangling (hydraulically entangled) process may be accomplished utilizing conventional hydroentangling equipment such as may be found, for example, in U.S. Pat. No. 3,485,706 to Evans. The hydroentangling of the present invention may be carried out with any appropriate working fluid such as, for example, water. Referring to FIG. 2, the working fluid flows through at least one manifold (130) which comprises one or more jets (132) (one shown) that evenly distributes the fluid to a series of individual holes or orifices (not shown). These holes or orifices may be from about 0.075 mm or from about 1 mm to about 0.18 mm or about 0.38 mm. The density of the jets may be from about 10 holes per cm, from about 12 holes per cm to about 24 holes per cm or to about 50 holes per cm. A single manifold may be used or more than one manifold may be arranged in succession such as the three shown in FIG. 1. In the hydroentangling process, the working fluid passes through the orifices at pressures ranging from about 200 pounds per square inch (psi), from about 500 psi, or from about 1000 psi to about 1400 psi, to about 2000 psi, to about 2900 psi, to about 3600 psi, to about 5000 psi or to about 6000 psi. The pressure may be controlled by appropriate control valves (not shown) and pressure gauges (not shown).

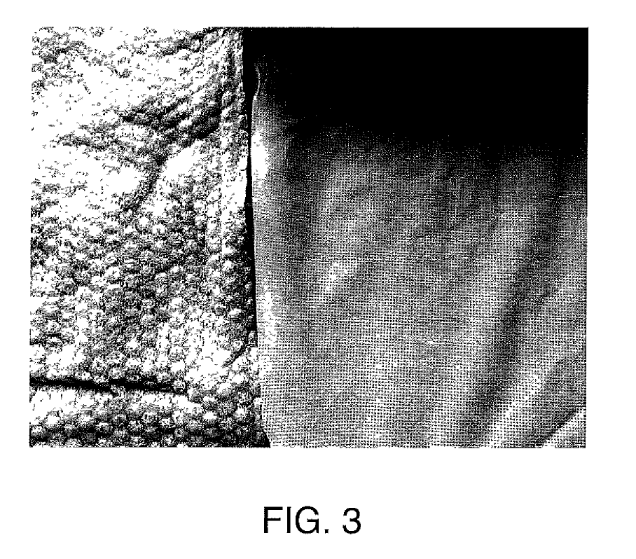

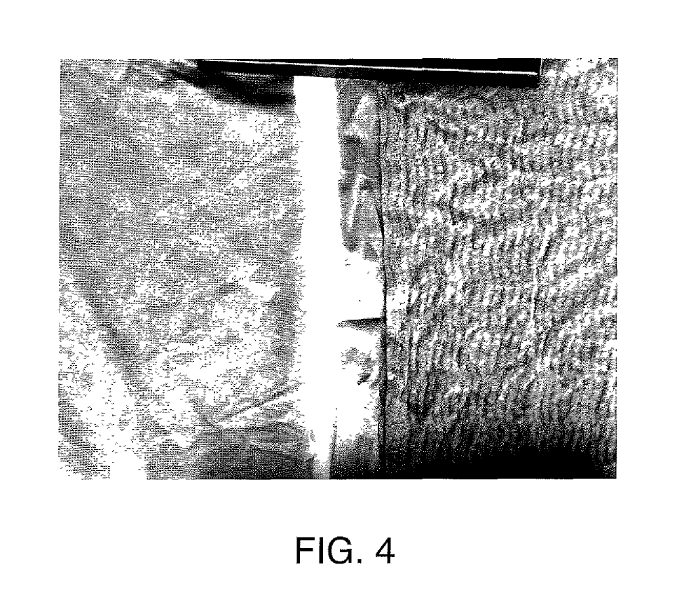

Referring to FIGS. 3 and 4, the present invention utilizes hydroentangling technology to pattern the SMS fabric laminates for a more aesthetically-pleasing and/or identifiable appearance and improved softness. The resulting material improves the overall perception of comfort and aesthetics for the end-user. With the use of a low-cost, lightweight material, it is important that the present invention is able to attain a visual definition of a pattern while still maintaining a high performing, high integrity material. The SMS fabric laminate used within the present invention, having an elevated content of meltblown fibers, is advantageous because the meltblown fibers are fine enough and abundant enough to allow the fluid from the jet strips to remove or displace the meltblown fibers without creating a hole or defect in the final SMS patterned laminate. The difference in fiber size between the large spunbond fibers and the lighter meltblown fibers creates the visible pattern that can be appreciated despite the lower basis weight of the overall SMS nonwoven laminate. Both FIGS. 3 and 4 show on the left of the images a typical SMS nonwoven laminate that does not have the attributes of the present invention and has not been through the process provided by the present invention. On the right of the images, the SMS nonwoven patterned laminates of the present invention uses a high level of meltblown, in combination with hydroentangling, to deliver a visible and pleasing pattern, although the basis weight of the overall SMS fabric laminate is low. The resulting visibly distinct appearance is a surface that may be desirable to many consumers. The difference between the patterns produced in FIG. 3 and FIG. 4 depends on the patterned screen used within the process which offers more variety to the end-user. In an absorbent article, for example, such patterns can create more variety options within a package of articles. For example, the SMS nonwoven laminate may be used as an outer cover material of an absorbent article. With a variety of visibly distinct patterns, varied absorbent articles, may be more desirable and offer an advantage to a consumer seeking more options in one package. For example, as incontinence articles become increasingly popular, it may be important to present a variety of articles with visibly distinct patterns without costly changes or other disadvantaged consumer impacts. Current options of absorbent articles are limited in variety. Diapers may be varied by costly graphics; however, the outer covers typically offer a standard nonwoven surface. Incontinence articles typically fail to offer a variety of graphics or nonwoven surfaces. Therefore, a need exists for offering the consumer a package that is able to provide a varied selection of articles wherein a low basis weight SMS nonwoven laminate with visibly distinct patterns is used as part of the construction of the article. The SMS nonwoven laminate of the present invention may be selected for use as a topsheet, backsheet, non-absorbent surge layer or as a facing for stretch laminates. Whether it is the outer cover, the topsheet or any other use of the like, the present invention provides opportunities for enhancing the variety of selecting SMS nonwoven laminates. The SMS nonwoven patterned laminate of the present invention may also be used as a wiper article, wet or dry, such as that used for floor wipes, hand wipes, baby wipes, feminine care wipes, industrial wipes or the like. Overall, the present invention provides a variety of visibly distinct patterns within an SMS nonwoven laminate without the costs usually necessary to achieve the results advantaged by the present invention.

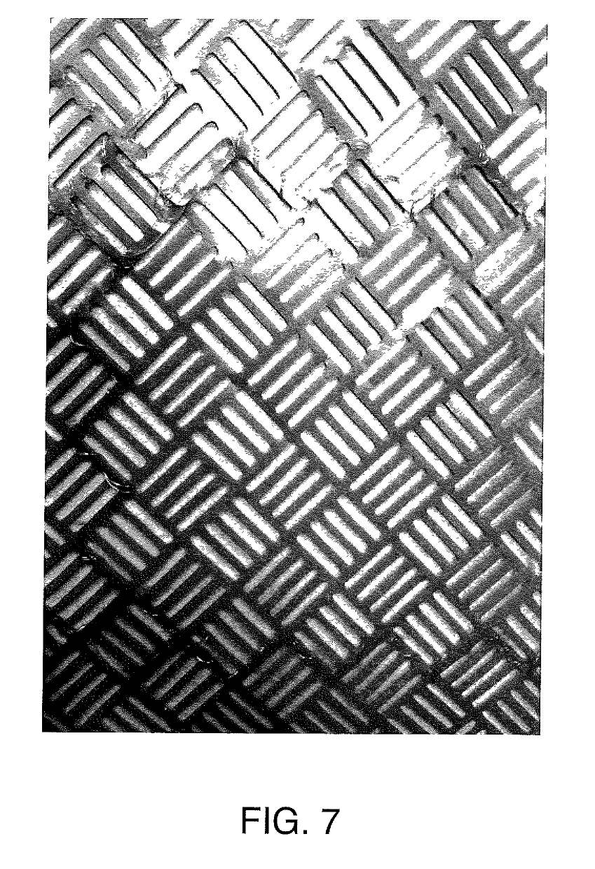

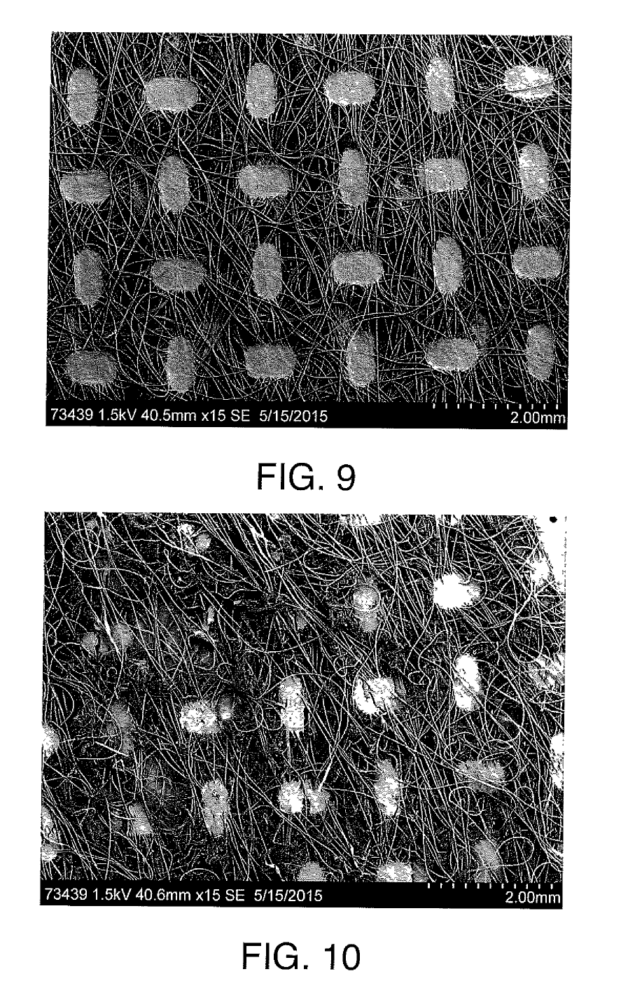

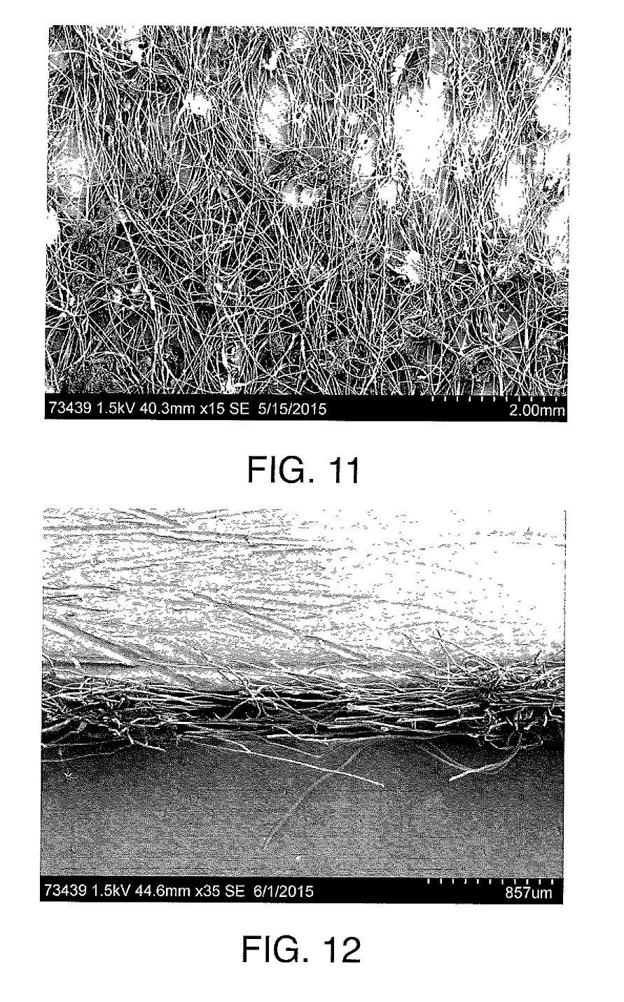

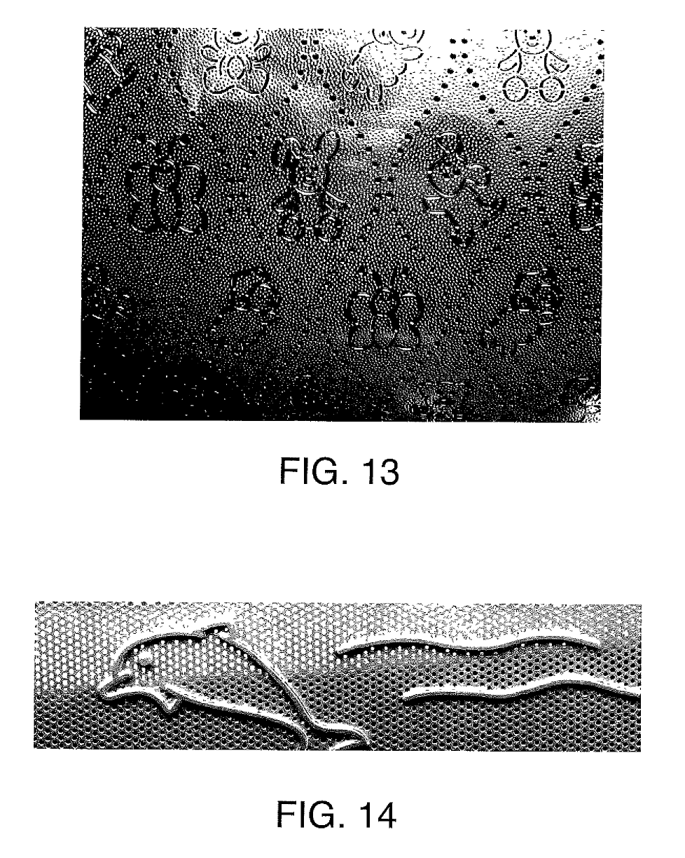

FIGS. 5, 7, 13 and 14 show a more detailed view of a portion of exemplary patterned screens that could be used within the process of the present invention. These screens may be used directly on the drum or roll or may be in other areas of the hydroentangling equipment where it may lay flat. Accordingly, FIGS. 6 and 8 show the resulting SMS nonwoven laminates that are produced as a result of the hydroentangling process of the present invention. In FIGS. 6, and 8, the areas where the meltblown fibers have been displaced are clearly visible and show fewer fibers since they have been removed or displaced to create the resulting visibly distinct pattern. The present invention and images shown are in contrast to the 17 gsm basis weight SMS nonwoven laminate of FIG. 9 where there is a very uniform dispersion of spunbond and meltblown fibers (bond points are seen as the oval dots). A 17 gsm basis weight SMS nonwoven laminate that has been hyrdoentangled by the process of the present invention provides visibly perceptible patterns and distinct surfaces such as that which can be seen in FIGS. 10 and 11. In FIG. 10, the hydrojets were used at a lower psi (1450) compared to that in FIG. 11 (3481 psi). Although FIG. 10 shows displacement of the meltblown fibers, the pattern is more visible with the increased pressure of the jets in FIG. 11. In FIG. 12, a magnified SEM image of a cross-sectional view of a 17 gsm basis weight SMS nonwoven laminate shows how the meltblown fibers have been displaced along the X-axis from the central focal point of the photo to the right and left of the material. The result would be similar to what is seen on the right sides of the images in FIGS. 3 and 4. Not only does the pressure of the jets during hydroentangling contribute to the overall resulting pattern, the basis weight of the SMS nonwoven laminate, the speed at which the line moves, the number of jets, positioning of the manifold, and the percentage of spunbond to meltblown fibers have an overall contribution to the resulting pattern. The present invention appreciates discovering the appropriate balance of all elements to successfully arrive at the SMS nonwoven laminate of the present invention.

For the hydroentangling, it is important to utilize pressures that will remove or displace the lighter sized meltblown fibers without causing a hole or disruption to the final SMS patterned composite. Thus, pressures should be monitored so as to avoid undue rupturing. The pressure of the jets (132) may also be adjusted to be relative to the speed at which the SMS fabric laminate is moved. The SMS fabric laminate may be processed at speeds of from about 350 feet per minute (fpm), from about 487 fpm, or from about 500 fpm, to about 1000 fpm, to about 1400 fpm, to about 1600 feet fpm, to about 1900 fpm or to about 2200 fpm. The SMS fabric laminate moves in the machine-direction, supported by the web carrier surface (110). The web carrier surface (110) may be for example, a single plane belt or a standard mesh screen. There may be one or multiple injectors/jet strips that are positioned typically from between about 0.005 m, from about 0.010 m, from about 0.015 m from about 0.025 m, to about 0.035 m to about 0.05 m above the SMS fabric laminate and patterned screen roll. The jets exhaust most of the kinetic energy to primarily rearrange the meltblown fibers within the SMS fabric laminate and to rebound and dissipate energy against the spunbond fibers. As shown in FIG. 2, a drum or roll (120) may be connected to a high pressure vacuum (122) to aid in removing used water and meltblown fibers from the SMS fabric laminate to prevent flooding of the resulting patterned SMS fabric composite. As shown in FIG. 1, the SMS patterned composite may be moved from a roll (120) to downstream where a dewatering vacuum (124) is used to further remove excess water from the laminate. The laminate is desirably transported downstream to a dryer mechanism (140) such as, for example, a dryer with an omega roll of 270° drying wrap angle or, for example, a through-air dryer, in which it is dried at temperatures up to the maximum non-destructive temperatures allowed by the SMS patterned laminate. Temperatures may vary according to the line speed, basis weight and pressure of the jets. Again, any typical drying operation is suitable and may be positioned downstream of the hydroentangling process.

While not shown, it may be desirable to use finishing steps and/or post treatment processes to impart selected properties to the hydroentangled composite. For example chemical post treatments may be added to the composite at a later step, or the composite may be transported to cutters, slitters or other processing equipment for converting the composite into a final product, such as wipes, components of personal care absorbent articles, or medical garment or covering fabrics.

The present invention may provide unique opportunities for packing personal care products, such as, for example, absorbent articles. Many absorbent articles may be placed in a package such as, for example, a polypropylene bag that combines a number of absorbent articles together for convenient purchasing. Any such container for articles may be used. The present invention provides for an absorbent article that is contained within a package with at least one other absorbent article comprising a SMS nonwoven laminate having a basis weight of less than about 20 gsm, said nonwoven laminate comprising outer layers of continuous spunbond filaments and an internal layer of from about 18% to about 43%, by weight of the nonwoven laminate of meltblown fibers, said nonwoven laminate having been hydroentangled to create visibly distinct patterns on the surface of at least one of the outer layers of said nonwoven laminate; and wherein prior to said hydroentangling, the content of meltblown fibers in the nonwoven laminate was from about 20% to about 45%, by weight of the nonwoven laminate; and wherein said package offers a variety of absorbent articles wherein at least one absorbent article differs from said other absorbent article because of the visibly distinct pattern on at least one surface of said other absorbent article. Thus, for example, the present invention may have an article with an SMS nonwoven laminate of the present invention comprising a visibly distinct pattern such as that shown in FIG. 6 in the same package with an article with an SMS nonwoven laminate of the present invention comprising a visibly distinct pattern such as that shown in FIG. 8. Having such a variety of articles in the same package offers a uniqueness that can only be appreciated by the attributes and advantageous of the present invention.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Example 1

An SMS nonwoven laminate having a basis weight of 17 gsm was hydroentangled according to the process of the present invention described herein. The SMS nonwoven laminate was processed through a hydroentangling equipment with a line speed of 50 fpm and utilized a patterned screen roll.

Two hydro jets were positioned above the roll and exerted a stream of water at a pressure of 3481 psi from the first hydro jet and 5076 psi from the second hydro jet to create a visibly distinct patterned SMS nonwoven laminate.

### Example 2

An SMS nonwoven laminate having a basis weight of 17 gsm was hydroentangled according to the process of the present invention described herein. The SMS nonwoven laminate was processed through a hydroentangling equipment with a line speed of 50 fpm and utilized a patterned screen roll. Two hydro jets were positioned above the roll and exerted a stream of water at a pressure of 3481 psi from the first hydro jet and 5076 psi from the second hydro jet to create a visibly distinct CD oriented patterned SMS nonwoven laminate.

### Example 3

An SMS nonwoven laminate having a basis weight of 17 gsm was hydroentangled according to the process of the present invention described herein. The SMS nonwoven laminate was processed through a hydroentangling equipment with a line speed of 50 fpm and utilized a patterned screen roll. Two hydro jets were positioned above the roll and exerted a stream of water at a pressure of 3481 psi from the first hydro jet and 5076 psi from the second hydro jet to create a visibly distinct MD oriented patterned SMS nonwoven laminate.

### Example 4

An SMS nonwoven laminate having a basis weight of 17 gsm was hydroentangled according to the process of the present invention described herein. The SMS nonwoven laminate was processed through a hydroentangling equipment with a line speed of 360 fpm and utilized a patterned screen roll. Two hydro jets were positioned above the roll and exerted a stream of water at a pressure of 3481 psi from the first hydro jet and 5076 psi from the second hydro jet to create a visibly distinct MD oriented patterned SMS nonwoven laminate.

### Example 5

An SMS nonwoven laminate having a basis weight of 15 gsm was hydroentangled according to the process of the present invention described herein. The SMS nonwoven laminate was processed through a hydroentangling equipment with a line speed of 50 fpm and utilized a patterned screen roll. Two hydro jets were positioned above the roll and exerted a stream of water at a pressure of 3481 psi from the first hydro jet and 5076 psi from the second hydro jet to create a visibly distinct MD oriented patterned SMS nonwoven laminate.

### Example 6

An SMS nonwoven laminate having a basis weight of 15 gsm was hydroentangled according to the process of the present invention described herein. The SMS nonwoven laminate was processed through a hydroentangling equipment with a line speed of 65 fpm and utilized a patterned screen roll. Two hydro jets were positioned above the roll and exerted a stream of water at a pressure of 2176 psi from the first hydro jet and 2176 psi from the second hydro jet to create a visibly distinct patterned SMS nonwoven laminate as shown in FIG. 6.

### Example 7

An SMS nonwoven laminate having a basis weight of 15 gsm was hydroentangled according to the process of the present invention described herein. The SMS nonwoven laminate was processed through a hydroentangling equipment with a line speed of 65 fpm and utilized a patterned screen roll. Two hydro jets were positioned above the roll and exerted a stream of water at a pressure of 2176 psi from the first hydro jet and 2176 psi from the second hydro jet to create a visibly distinct patterned SMS nonwoven laminate as shown in FIG. 8.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A SMS nonwoven laminate having a basis weight of less than about 20 gsm, said nonwoven laminate comprising outer layers of continuous spunbond filaments and an internal layer of from about 18% to about 43%, by weight of the nonwoven laminate of meltblown fibers, said nonwoven laminate having been hydroentangled to create visibly distinct patterns on the surface of at least one of the outer layers of said nonwoven laminate; and wherein prior to said hydroentangling, the content of meltblown fibers in the nonwoven laminate was from about 20% to about 45%, by weight of the nonwoven laminate.

2. The nonwoven laminate of claim 1 wherein the visibly distinct pattern is three-dimensional.

3. The nonwoven laminate of claim 1 wherein the spunbond filaments have diameters of from about 10 microns to about 30 microns.

4. The nonwoven laminate of claim 1 wherein the hydroentangling utilizes water at a pressure of from about 200 psi to about 6000 psi.

5. The nonwoven laminate of claim 1 wherein the hydroentangling comprises a patterned screen, said patterned screen selected from a flat wire mesh, three-dimensional wire mesh, or stencil-like metal surface with a variety of openings and closings.

6. The nonwoven laminate of claim 5 wherein the patterned screen is on a flatbed belt, roll or combinations thereof.

7. The nonwoven laminate of claim 6 wherein the patterned screen is on a roll and at least one manifold comprising at least one jet strip is positioned from about 0.005 m to about 0.05 m above the nonwoven laminate and said roll.

8. The nonwoven laminate of claim 1 wherein the basis weight is from about 15 gsm to about 17 gsm.

9. The nonwoven laminate of claim 1 wherein said internal layer comprises from about 26% to about 28%, by weight of the nonwoven laminate of meltblown fibers and prior to said hydroentangling the content of meltblown fibers in the nonwoven laminate was from about 28% to about 30%, by weight of the nonwoven laminate.

10. The nonwoven laminate of claim 1 wherein said laminate is selected for use as a wiper article, a personal care product or an absorbent medical product.

11. The nonwoven laminate of claim 10 wherein the personal care product is a topsheet, backsheet, non-absorbent surge layer or facing for stretch laminates.

12. The personal care product of claim 10 wherein the product is an absorbent article comprising a SMS nonwoven laminate having a basis weight of less than about 20 gsm, said nonwoven laminate comprising outer layers of continuous spunbond filaments and an internal layer of from about 18% to about 43%, by weight of the nonwoven laminate of meltblown fibers, said nonwoven laminate having been hydroentangled to create visibly distinct patterns on the surface of at least one of the outer layers of said nonwoven laminate; and wherein prior to said hydroentangling, the content of meltblown fibers in the nonwoven laminate was from about 20% to about 45%, by weight of the nonwoven laminate.

13. The absorbent article of claim 12 wherein the basis weight of the nonwoven laminate is from about 15 gsm to about 17 gsm.

14. The absorbent article of claim 12 wherein the content of meltblown fibers is from about 28% to about 30%, by weight of the nonwoven laminate.

15. The absorbent article of claim 12 wherein the visibly distinct pattern is three-dimensional.

16. A method of producing a SMS nonwoven laminate having a visibly distinct pattern and a basis weight of less than about 20 gsm, said method comprising the steps of:
a. providing, in the machine direction to a hydroentangling equipment, a nonwoven laminate having a basis weight of from about 5 gsm to about 20 gsm, said nonwoven laminate comprising outer layers of continuous spunbond filaments and an internal layer of from about 20% to about 45%, by weight of the nonwoven laminate of meltblown fibers, to a web carrier surface of said hydroentangling equipment;
b. continuing said nonwoven laminate on to a patterned screen roll wherein at least one manifold, of the hydroentangling equipment, comprises at least one water pressure jet positioned from about 0.005 m to about 0.05 m above said laminate and patterned screen roll;
c. hydroentangling said nonwoven laminate with said jet at a water pressure of from about 200 psi to about 6000 psi;
d. continuing said nonwoven laminate to a dryer;
e. providing a finished nonwoven laminate material having a basis weight of from about 5 gsm to about 20 gsm, wherein said nonwoven laminate material comprises outer layers of continuous spunbond filaments and an internal layer of from about 18% to about 43%, by weight of the nonwoven laminate of meltblown fibers, and wherein the surface of said nonwoven laminate material has a visibly distinct pattern as a result of the hydroentangling on the surface of at least one of the outer layers of said nonwoven laminate.

17. The method of claim 16 wherein the speed of the web carrier surface is about 350 fpm to about 2200 fpm.

18. The method of claim 16 wherein the orifices of said jets are from about 0.075 mm to about 0.38 mm.

19. The method of claim 16 wherein the density of said orifices are from about 10 holes/cm to about 50 holes/cm.

20. The method of claim 16 wherein the visibly distinct pattern is three-dimensional.

21. The absorbent article of claim 12 wherein the absorbent article is contained within a package with at least one other absorbent article comprising a SMS nonwoven laminate having a basis weight of less than about 20 gsm, said nonwoven laminate comprising outer layers of continuous spunbond filaments and an internal layer of from about 18% to about 43%, by weight of the nonwoven laminate of meltblown fibers, said nonwoven laminate having been hydroentangled to create visibly distinct patterns on the surface of at least one of the outer layers of said nonwoven laminate; and wherein prior to said hydroentangling, the content of meltblown fibers in the nonwoven laminate was from about 20% to about 45%, by weight of the nonwoven laminate; and wherein said package offers a variety of absorbent articles wherein at least one absorbent article differs from said other absorbent article because of the visibly distinct pattern on at least one surface of said other absorbent article.
